(19) United States
(12) Patent Application Publication
(10) Pub. No.: US 2024/0225698 A1
(43) Pub. Date: Jul. 11, 2024

(54) EXTERNAL FIXATION K-WIRE CLAMP AND CORRESPONDING METHOD OF USE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Scott Smyth, Memphis, TN (US); Steve Miller, Scotts Valley, CA (US); Steven Moran, Rochester, MN (US)

(21) Appl. No.: 18/521,352

(22) Filed: Nov. 28, 2023

Related U.S. Application Data
(60) Provisional application No. 63/437,827, filed on Jan. 9, 2023.

(51) Int. Cl. A61B 17/64

(52) U.S. Cl. CPC A61B 17/6425; A61B 17/6458; A61B 17/6475; A61B 2017/606

(57) ABSTRACT

An external fixation K-wire clamp including an elongated channel arranged and configured to receive one or more K-wires. In some examples, the elongated channel is devoid of any predefined grooves so that the one or more K-wires can be positioned anywhere along the length of the elongated channel and/or in various orientations. In addition, and/or alternatively, an exemplary method of use for treating a distal radius fracture is disclosed.

### CROSS-REFERENCE TO RELATED APPLICATIONS

This is a non-provisional of, and claims the benefit of the filing date of, U.S. provisional patent application No. 63/437,827, filed Jan. 9, 2023, entitled “External Fixation K-Wire Clamp and Corresponding Method of Use,” the entirety of which application is incorporated by reference herein.

### FIELD OF THE DISCLOSURE

The present disclosure relates generally to orthopedic devices, systems, and methods for facilitating fracture alignment, and more particularly to an external fixation clamp arranged and configured to secure a Kirschner Wire (e.g., a K-wire).

### BACKGROUND OF THE DISCLOSURE

People suffer bone fractures each year. In many instances, a person that suffers a bone fracture is required to use a bone alignment device such as, for example, an external fixation system to align two or more bones, bone fragments, bone pieces, etc. (terms used interchangeably herein without the intent to limit or distinguish). For example, thousands of injuries are reported annually with patients having complex or comminuted fractures of the distal radius, ulna, and carpals/metacarpals. Fractures of this type often occur due to high-energy traumatic events such as, for example, car accidents, falls, or sporting events.

When such cases are presented at the hospital, physicians must often decide between non-surgical and surgical treatment options, which often are dependent on the severity of the injury. Non-surgical treatment generally includes applying a cast to the patient's wrist to stabilize and hold the patient's anatomy in-place during healing. More severe fractures and injuries, however, often involve displacement of bone, which requires a surgical approach to restore proper anatomical alignment. To accomplish this, the physician may need to manipulate the displaced bone. Once the bone has been properly reset, the bone is fixed in place using, for example, one or more bone fixation plates or an external fixation system or construct. In use, the external fixation system or construct can be used to either permanently fix the patient's bone during healing or temporarily hold the patient's bone until such time that a permanent bone fixation plate can be implanted. Thus, if a surgical treatment is required, typically the treating physician can either implant an internal bone fixation plate such as, for example, a volar plate, or couple an external fixation system or construct, which can remain in place during the entire healing period or temporarily until an internal bone fixation plate can be implanted. As a result, in use, external fixation systems or constructs can be used to stabilize a patient's bone either as a permanent or temporary treatment option.

Generally speaking, external fixation systems or constructs utilize a plurality of components that physicians can use to couple patient's bones. Constructs may include, for example, one or more spanning rods or bars (terms used interchangeably herein without the intent to limit), a plurality of threaded half-pins used to engage a patient's bone, one or more rod-to-rod clamps used to intercouple spanning rods together, and one or more rod-to-pin clamps, also referred to as multi-pin clamps, used to couple the threaded half-pins to the spanning rod. In use, physicians may also utilize one or more Kirschner Wires (e.g., K-wires) to manipulate patient's bones.

For example, referring to FIG. 1, an example of a known rod-to-rod clamp 10 is shown. As illustrated, the rod-to-rod clamp 10 includes first and second mini-clamps 12 coupled by a threaded bolt and nut assembly 14. In use, each mini-clamp 12 includes a channel 16 arranged and configured to receive a spanning rod therein. As will be appreciated by one of ordinary skill in the art, in the unlocked position, the mini-clamps 12 can be swiveled or rotated to provide the physician with flexibility when coupling to the spanning rod. In use, tightening of the threaded bolt and nut assembly 14 fixes the position of the mini-clamps 12 relative to each other and secures the position of the spanning rod within the channel 16.

Referring to FIG. 2, an example of a known rod-to-pin clamp 20 is shown. As illustrated, the rod-to-pin clamp 20 includes a rod-attachment clamp 22 including a channel 24 arranged and configured to receive the spanning rod therein and a pin clamp 26 arranged and configured to receive one or more threaded half-pins therein. As illustrated, the pin clamp 26 includes a plurality of spaced-apart grooves, recesses, channels, etc. 28 for receiving the threaded half-pins. That is, as will be readily appreciated by one of ordinary skill in the art, the rod-to-pin clamp 20 includes a plurality of grooves 28 for securing one or more parallel threaded half-pins, which may be implanted into a patient's bone.

Referring to FIG. 3, an example of a known K-wire clamp 30 is shown. As illustrated, the K-wire clamp 30 includes a rod-attachment clamp 32 including a channel 34 arranged and configured to receive the spanning rod 40 therein and a K-wire clamp 36 arranged and configured to receive one or more K-wires therein. As illustrated, the K-wire clamp 36 includes a plurality of spaced-apart grooves, recesses, channels, etc. 38 for receiving the K-wires 42. That is, as will be readily appreciated by one of ordinary skill in the art, the K-wire clamp 30 includes a plurality of grooves 38 for securing one or more parallel K-wires 42, which may be implanted into a patient's bone.

One drawback of existing external fixation clamps is their inability to integrate both threaded half-pins and multiple K-wires at non-parallel angles into the same external fixation system or construct. As a result, external fixation of the distal radius is often only performed to temporarily stabilize the patient's anatomy until an additional, second surgical procedure can be performed to implant a permanent fixation solution such as, for example, implantation of an internal bone fixation plate. In return, this further increases the likelihood of fracture site infection.

It would be beneficial to provide an external fixation clamp that is capable of securing, affixing, or the like one or more K-wires at various relative angles. Specifically, it would be beneficial to provide an external fixation clamp that provides physicians with greater flexibility in positioning the one or more K-wires relative to the K-wire clamp. It is with respect to this and other considerations that the present disclosure may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

An external fixation K-wire clamp is disclosed. In use, the external fixation K-wire clamp is arranged and configured to be used in an external fixation system including, inter alia, a spanning rod, a K-wire, optional rod-to-rod clamps, rod-to-pin clamps, threaded pins, etc.

In some examples, the external fixation K-wire clamp includes an elongated channel configured to receive a K-wire, the external fixation K-wire clamp being devoid of any predefined grooves for receiving the K-wire (e.g., the contacting or abutting surfaces defining the elongated channel do not include any predefined grooves for receiving the K-wire) so that the K-wire can be positioned anywhere along a length of the elongated channel and/or can be orientated in various orientations in the plane of the elongated channel, such as, for example, converging pins with one running from lateral to medial and another running posterior to anterior (e.g., the K-wire can be orientated in any one of a plurality of different orientations). For example, the external fixation K-wire clamp is configured to receive and secure one or more K-wires that are positioned non-parallel to the contacting or abutting surfaces defining the elongated channel.

In any preceding or subsequent example, the contacting or abutting surfaces of the external fixation K-wire clamp may be configured to simultaneously receive and secure the various positions of a plurality of K-wires inserted therein. In use, the plurality of K-wires can be parallel to each other. Alternatively, the plurality of K-wires can be angled relative to each other (e.g., non-parallel) so long as the K-wires do not overlap with each other.

In any preceding or subsequent example, wherein the external fixation K-wire clamp includes a main body including a first contacting or abutting surface and a mechanism for coupling the external fixation K-wire clamp to a spanning rod, a plate member including a second contacting or abutting surface parallel to the first contacting surface, and a mechanism for moving the plate member relative to the main body to selectively secure the position of a K-wire between the contacting or abutting surfaces (e.g. within an elongated channel between the first and second contacting surfaces).

In any preceding or subsequent example, the mechanism for coupling to the spanning rod comprises a mini-rod clamp including a threaded bolt arranged and configured to threadably engage a threaded opening formed in the external fixation K-wire clamp (e.g., the external fixation K-wire clamp includes a threaded bore arranged and configured to receive a threaded stem or bolt associated with a mini-rod clamp for coupling the external fixation K-wire clamp to a spanning rod).

In any preceding or subsequent example, wherein the mechanism for moving the plate member relative to the main body includes a plurality of fasteners (e.g., screws) coupling the plate member to the main body.

In any preceding or subsequent example, wherein the external fixation K-wire clamp includes a plurality of biasing members positioned about the plurality of fasteners, respectively, in-between the plate member and the main body for biasing the plate member away from the main body. In any preceding or subsequent example, the biasing members are a compression or coiled spring.

In any preceding or subsequent example, the contacting or abutting surfaces of the external fixation K-wire clamp may include a roughened, bead blasted, or diamond knurled surfaces.

A method of treating a distal radius fracture is also disclosed. The method including inserting one or more proximal pins into a patient's radius, inserting one or more distal pins into a patient's carpal or metacarpal bone such that the fracture is positioned in-between the one or more proximal pins and the one or more distal pins, coupling the one or more proximal pins and the one or more distal pins to a spanning rod, the spanning rod extending across the fracture, inserting a K-wire into a fracture site including the fracture, coupling an external fixation K-wire clamp to the spanning rod and to the inserted K-wire, securing the K-wire to the external fixation K-wire clamp, and removing, post-healing, the one or more distal pins, the one or more proximal pins, the K-wire, and the spanning rod.

In any preceding or subsequent example, wherein the one or more proximal pins are coupled to the spanning rod using a proximal rod-to-pin clamp. The one or more distal pins are coupled to the spanning rod using a distal rod-to-pin clamp.

In any preceding or subsequent example, a plurality of K-wires are inserted, wherein the external fixation K-wire clamp is configured to simultaneously secure positions of the plurality of K-wires.

In any preceding or subsequent example, at least one of the plurality of K-wires is non-parallel to a second one of the plurality of K-wires.

In any preceding or subsequent example, wherein the plurality of K-wires are not in a plane that is parallel to the spanning rod.

Examples of the present disclosure provide numerous advantages. For example, by providing an external fixation K-wire clamp that can secure a K-wire including in a non-parallel configuration, physicians are provided with increased flexibility during construct assembly. In addition, by enabling the K-wire to be fixedly secured to the external fixation system or construct, increased stiffness can be provided. In addition, and/or alternatively, by utilizing an external fixation system or construct to treat a patient's distal radius fracture, definitive treatment can be achieved in severe high-energy fractures without requiring additional surgery to implant a bone fixation plate (e.g., a volar plating system).

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 illustrates a perspective view of a conventional rod-to-rod clamp;

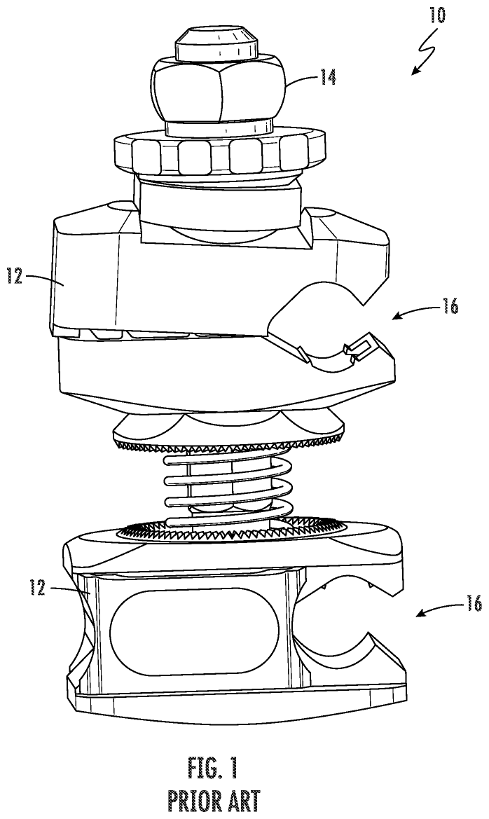

FIG. 2 illustrates a perspective view of a conventional rod-to-pin clamp;

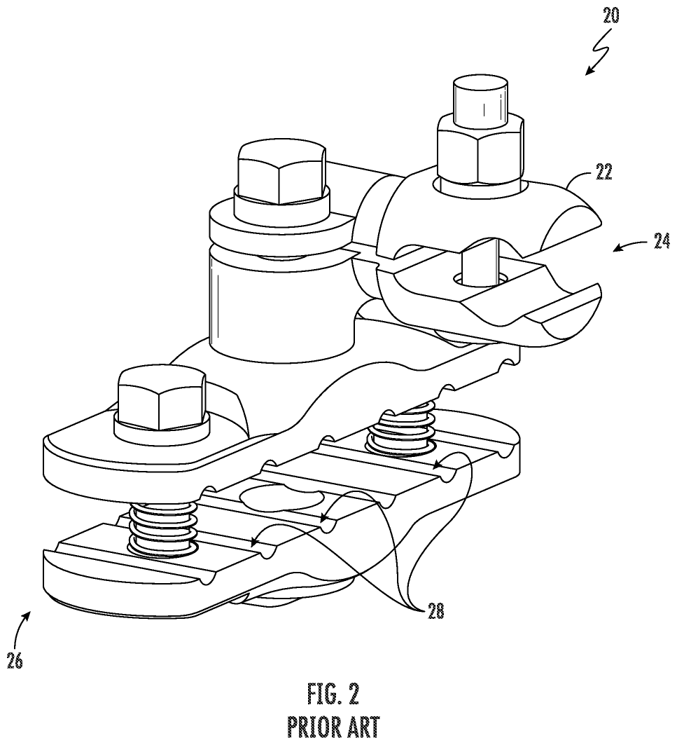

FIG. 3 illustrates a perspective view of a conventional external fixation construct including multiple K-wire clamps;

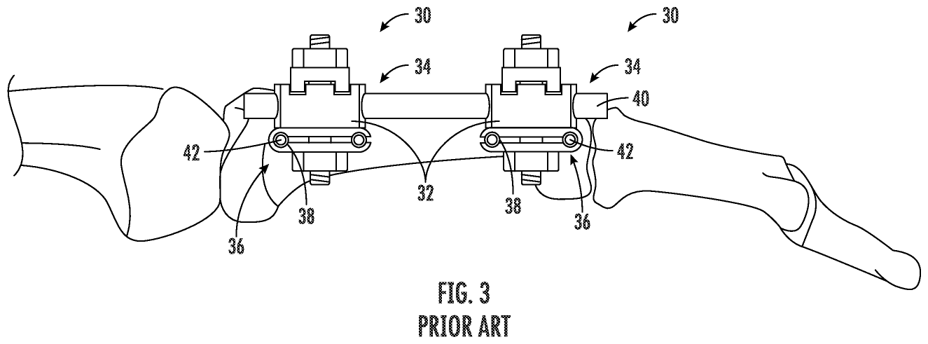

FIG. 4 illustrates a perspective view of an example of an external fixation K-wire clamp in accordance with one or more features of the present disclosure;

FIG. 5 illustrates a top perspective view of an external fixation system or construct including the external fixation K-wire clamp shown in FIG. 4, the external fixation system or construct being used to affix a distal radius fracture in accordance with one or more features of the present disclosure;

FIG. 6 is a flowchart of an example surgical technique used to affix and treat a patient's distal radius fracture in accordance with one or more features of the present disclosure.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of an external fixation clamp or a K-wire clamp (referred to herein as an external fixation K-wire clamp) will now be described more fully herein with reference to the accompanying drawings, in which one or more features of the external fixation K-wire clamp will be shown and described. It should be appreciated that the various features may be used independently of, or in combination with, each other. It will be appreciated that the external fixation K-wire clamp as disclosed herein may be embodied in many different forms and may selectively include one or more concepts, features, or functions described herein. As such, the external fixation K-wire clamp should not be construed as being limited to the specific examples set forth herein. Rather, these examples are provided so that this disclosure will convey certain features of the external fixation K-wire clamp to those skilled in the art.

In accordance with one or more features of the present disclosure, an external fixation K-wire clamp includes an elongated slot or channel arranged and configured to receive a K-wire. In use, contrary to conventional external fixation clamps such as, for example, conventional K-wire clamps that include one or more predefined grooves for receiving K-wires, the elongated channel of the external fixation K-wire clamp of the present disclosure is devoid of any recesses, grooves, channels, etc. (e.g., the abutting surfaces do not include any predefined grooves for receiving the K-wire). Thus arranged, the K-wire can be positioned anywhere along the length of the elongated channel. In addition, and/or alternatively, the elongated channel enables the K-wire to be orientated in any orientation such as, for example, angled relative to the contacting or abutting surfaces of the elongated channel of the external fixation K-wire clamp. That is, in use, the external fixation K-wire clamp is configured to grip a K-wire anywhere along the length of the elongated channel in any number of different orientations. As such, the physician is provided with increased flexibility in constructing the external fixation system or construct.

In addition, and/or alternatively, in accordance with one or more features of the present disclosure, the external fixation K-wire clamp can be used in an external fixation system or construct that spans the patient's wrist (e.g., the external fixation K-wire clamp can be used in an external fixation system or construct used to treat a patient's distal radius fracture). In use, the external fixation system or construct includes a plurality of clamps such as, for example, rod-to-pin clamps, to secure the patient's radius and the patient's carpal or metacarpal bones (e.g., the external fixation system or construct includes first and second clamps to secure the proximal and distal sides of a patient's fracture). Thereafter, an external fixation K-wire clamp can be coupled to the construct and a K-wire can be used to secure or couple to one of the patient's bones in the wrist (e.g., metacarpal bones, fracture fragments of the distal radius). The patient's bone can be reset to its proper position and the position of the K-wire can be secured to the construct.

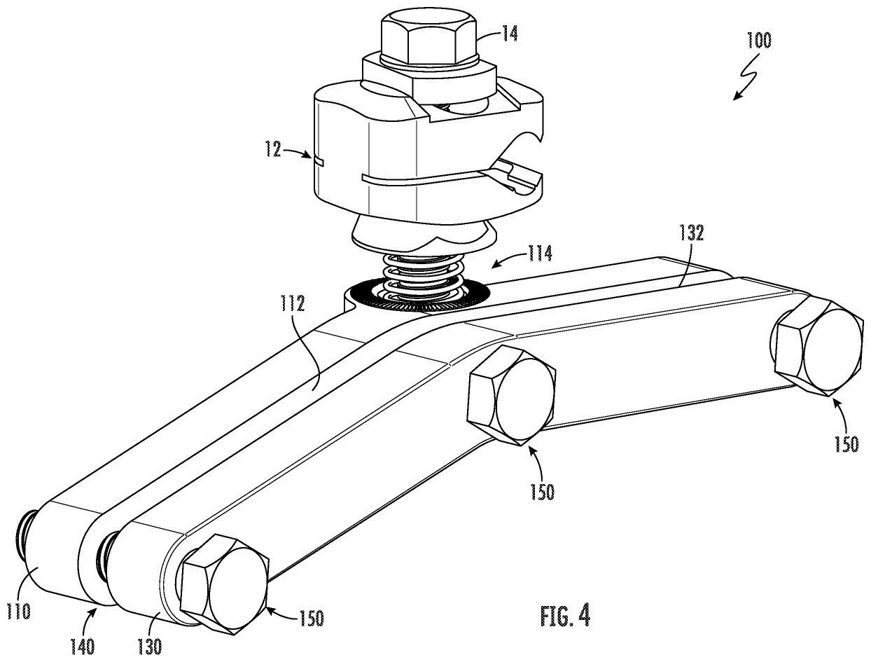

With reference to FIG. 4, an example of an external fixation K-wire clamp 100 in accordance with one or more features of the present disclosure is shown. As illustrated, the external fixation K-wire clamp 100 includes a main body 110 and a plate member 130. In use, the plate member 130 is coupled to the main body 110. In addition, as will be described in greater detail below, the plate member 130 is moveable relative to the main body 110 to define an elongated channel 140 between the main body 110 and the plate member 130 to selectively enable one or more K-wires to be secured within the elongated channel 140 between the plate member 130 and the main body 110.

The plate member 130 may be coupled to the main body 110 via any suitable connection mechanism now known or hereafter developed. In some examples, as illustrated, the plate member 130 may be coupled to the main body 110 via fasteners (e.g., threaded screws) 150. For example, as illustrated, in some examples, the plate member 130 may be coupled to the main body 110 via a plurality of fasteners 150 spaced along a length of the plate member 130 and main body 110. In use, the plate member 130 may include non-threaded openings to enable the fasteners 150 to pass freely therethrough while the main body 110 includes threaded screw holes for threadably engaging the fasteners 150. It should be appreciated that while the external fixation K-wire clamp 100 has been shown with three fasteners, more or less fasteners may be used (e.g., the location and quantity of fasteners along the clamp can be changed to change the balance of clamping force).

In some examples, biasing members such as, for example, compression springs, coil springs, or the like, may be positioned about the fasteners 150 between the plate member 130 and the main body 110. Thus arranged, the biasing members bias the plate member 130 away from the main body 110 when the fasteners 150 are not tightened (e.g., an unlocked position) to maintain the elongated channel 140 opened. Thereafter, once the K-wire has been properly implanted, the fasteners 150 can be rotated to move (e.g., compress) the plate member 130 against the main body 110 to secure the K-wire between the plate member 130 and the main body 110.

As illustrated, the main body 110 includes an abutting or contacting surface 112 (terms used interchangeably herein without the intent to limit or distinguish). Similarly, the plate member 130 includes a contacting surface 132. In use, the contacting surface 112 of the main body 110 and the contacting surface 132 of the plate member 130 are parallel to each other. Thus arranged, in accordance with the various features of the present disclosure, the external fixation K-wire clamp 100 can be used to affix one or more K-wires.

In addition, and/or alternatively, in accordance with one or more features of the present disclosure, the contacting surfaces 112, 132 of the main body 110 and the plate member 130 are devoid of any grooves for predefining areas for receiving the K-wire. Thus arranged, the elongated channel 140 defined by the space between the contacting surfaces 112, 132 can receive one or more K-wires including non-parallel K-wires (e.g., K-wires may be orientated or angled relative to the contacting surfaces 112, 132). For example, the elongated channel 140 can receive one or more K-wires anywhere along a length of the channel 140 and/or can receive one or more K-wires in any orientation so long as, if a plurality of K-wires are utilized, the K-wires do not overlap each other within the elongated channel 140. For example, as illustrated, the K-wire can be randomly orientated in a plane that is non-parallel to a spanning rod of an external fixation construct or system as will be described in greater detail below.

In addition, in some examples, the elongated channel 140 defined by the space between the contacting surfaces 112, 132 can simultaneously receive and secure the various positions of a plurality of K-wires inserted therein. In particular, the external fixation K-wire clamp 100 enables simultaneous fixation of a plurality of K-wires within the elongated channel 140 even if the K-wires are non-parallel with each other so long as, if a plurality of K-wires are utilized, the K-wires do not overlap each other within the elongated channel 140. That is, in use, the external fixation K-wire clamp 100 is configured to grip a plurality of K-wires anywhere along the length of the elongated channel 140 with any number of different orientations including non-parallel as long as the K-wires do not overlap with each other. As such, the physician is provided with increased flexibility in constructing the external fixation system or construct.

As illustrated, the external fixation K-wire clamp 100 includes a threaded bore 114 arranged and configured to receive a threaded bolt or stem associated with a bar clamp such as, for example, mini rod clamp 12. Thus, a mini rod clamp 12 may be threadably coupled to the external fixation K-wire clamp 100. For example, as illustrated, the external fixation K-wire clamp 100 may include an offset threaded bore 114 for receiving a threaded screw associated with the mini rod clamp 12. Thus arranged, a mini rod clamp 12 can be integrated into the external fixation K-wire clamp 100 to enable the external fixation K-wire clamp 100 to connect to a spanning rod of an external fixation system or construct without requiring a separate rod-to-rod clamp.

In accordance with one or more features of the present disclosure, a method 400 of using an external fixation K-wire clamp 100 in a distal radius fracture will now be described. It should be appreciated, that while the external fixation K-wire clamp 100 will be shown and described in connection with affixing a K-wire to a patient's hand or wrist (e.g., distal radius fractures), it is envisioned that the external fixation K-wire clamp 100 can be used in other locations such as, for example, in connection with small bones in the patient's foot.

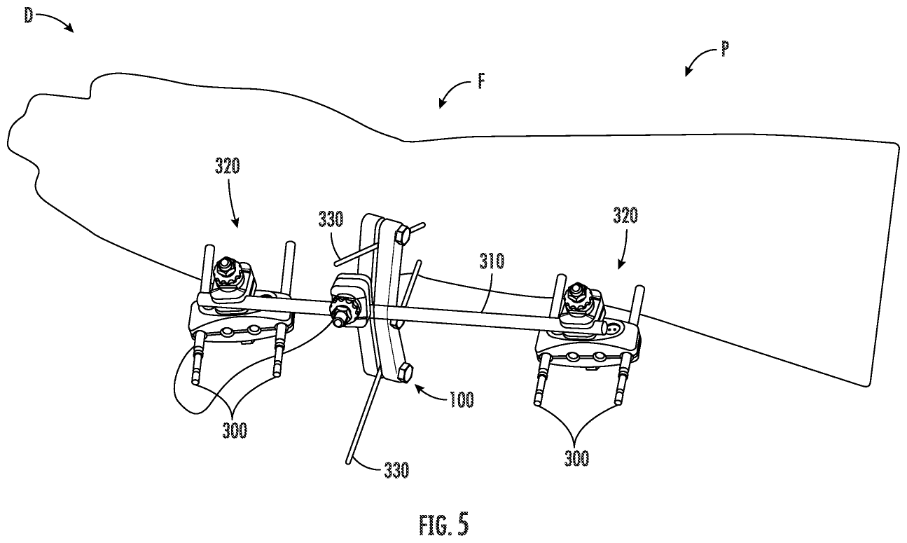

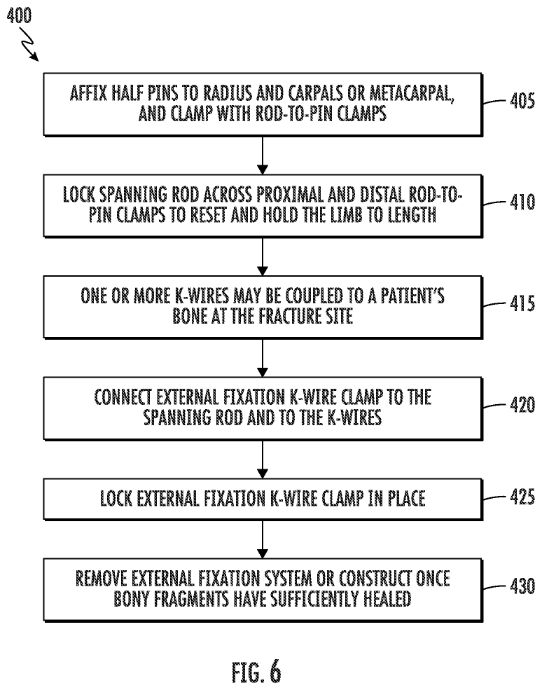

With reference to FIGS. 5 and 6, in accordance with known surgical procedures, at step 405, threaded half-pins 300 may be inserted into the patient's bones on either side of the fracture site F. For example, as illustrated, threaded half-pins 300 may be inserted into the patient's radius and into the patient's carpals or metacarpal bones (e.g., threaded half-pins 300 may be implanted into the dorsal and radial sides of a patient's wrist/hand). Thereafter, the threaded half-pins 300 may be coupled with conventional rod-to-pin clamps such as, for example, rod-to-pin clamps 320. Next, at step 410, a spanning rod 310 may be coupled to each of the rod-to-pin clamps 320. Thus arranged, the spanning rod 310 and rod-to-pin clamps 320 form an external fixation system or construct used to reset and hold the patient's limb to length. As illustrated, the spanning rod 310 extends across the fracture site F. Thus, in use, conventional rod-to-pin clamps 320 can be secured to the proximal P and distal D sides of the fracture site F using conventional half-pins 300 (e.g., 3-4 mm half-pins). A spanning rod 310 can then be used to couple the proximal and distal rod-to-pin clamps 320.

In accordance with one or more features of the present disclosure, at step 415, one or more K-wires 330 may be coupled to a patient's bone at the fracture site. Next, at step 420, the K-wires 330 may be coupled to the spanning rod 310 via the external fixation K-wire clamp 100. At step 425, the external fixation K-wire clamp 100 can be locked to secure the one or more K-wires 330 within the elongated channel 140 between the main body 110 and the plate member 130 of the external fixation K-wire clamp 100. As such, one or more K-wires 330 can be integrated with the external fixation system or construct. Finally, at step 430, the external fixation system or construct can be removed once the patient's bones have sufficiently healed.

As previously described, in use, a conventional mini rod clamp 12 or other suitable mechanism can be used to couple the external fixation K-wire clamp 100 to the spanning rod 310 in between the proximal and distal rod-to-pin clamps 320. In use, by providing adjoining contacting surfaces 112, 132 devoid of any predefined grooves for wire placement, the external fixation K-wire clamp 100 enables one or more K-wires 330 to be inserted and secured therein. That is, one or more K-wires 330 can be inserted into, for example, the patient's radiocarpal bones. Thereafter, the K-wires 330 can be locked in place by the external fixation K-wire clamp 100.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one,” “one or more,” and “and/or” as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

We claim:

1. An external fixation system comprising:
an external fixation K-wire clamp including:
a main body including a first contacting surface and a threaded bore arranged and configured to receive a threaded bolt associated with a mini-rod clamp to couple the external fixation K-wire clamp to a spanning rod;
a plate member including a second contacting surface parallel to the first contacting surface; and
a mechanism for moving the plate member relative to the main body to selectively secure a position of a K-wire within an elongated channel between the first and second contacting surfaces;
wherein the first and second contacting surfaces are devoid of any predefined grooves for receiving the K-wire.

2. The external fixation system of claim 1, further comprising a K-wire, wherein the K-wire can be positioned within the elongated channel anywhere a length of the elongated channel.

3. The external fixation system of claim 2, wherein the K-wire can be orientated in any one of a plurality of different orientations.

4. The external fixation system of claim 1, wherein the mechanism for moving the plate member relative to the main body includes a plurality of fasteners for coupling the plate member to the main body.

5. The external fixation system of claim 4, wherein the external fixation K-wire clamp further comprises a plurality of biasing members positioned about the plurality of fasteners, respectively, the plurality of biasing members positioned in-between the plate member and the main body for biasing the plate member away from the main body.

6. The external fixation system of claim 5, wherein the plurality of biasing members are a compression or coiled spring.

7. The external fixation system of claim 1, wherein the first and second contacting surfaces each include a roughened surface.

8. The external fixation system of claim 1, wherein the external fixation K-wire clamp further includes a mini-rod clamp including a threaded bolt to threadably engage the threaded bore formed in the main body of the external fixation K-wire clamp.

9. The external fixation system of claim 8, further including a spanning rod, the mini-rod clamp including a channel to couple the external fixation K-wire clamp to the spanning rod.

10. The external fixation system of claim 9, further comprising first and second rod-to-pin clamps, each of the first and second rod-to-pin clamps including a channel to couple the rod-to-pin clamp to the spanning rod, each of the first and second rod-to-pin clamps including a second channel for gripping threaded pins.

11. An external fixation system comprising:
a K-wire; and
an external fixation K-wire clamp including:
a main body including a first contacting surface and a threaded bore arranged and configured to receive a threaded bolt associated with a mini-rod clamp to couple the external fixation K-wire clamp to a spanning rod;
a plate member including a second contacting surface parallel to the first contacting surface; and
a mechanism for moving the plate member relative to the main body to selectively secure a position of a K-wire within an elongated channel between the first and second contacting surfaces;
wherein:
the first and second contacting surfaces are devoid of any predefined grooves for receiving the K-wire; and
the K-wire can be positioned within the elongated channel anywhere a length of the elongated channel; and/or at any one of a plurality of different orientations.

12. The external fixation system of claim 11, wherein the mechanism for moving the plate member relative to the main body includes a plurality of fasteners for coupling the plate member to the main body.

13. The external fixation system of claim 12, wherein the external fixation K-wire clamp further comprises a plurality of biasing members selected from one of a compression spring and a coiled spring, the biasing members positioned about the plurality of fasteners, respectively, the plurality of biasing members positioned in-between the plate member and the main body for biasing the plate member away from the main body.

14. The external fixation system of claim 11, wherein the first and second contacting surfaces each include a roughened surface.

15. The external fixation system of claim 11, wherein the external fixation K-wire clamp further includes a mini-rod clamp including a threaded bolt to threadably engage the threaded bore formed in the main body of the external fixation K-wire clamp.

16. The external fixation system of claim 15, further including a spanning rod, the mini-rod clamp including a channel to couple the external fixation K-wire clamp to the spanning rod.

17. A method of treating a distal radius fracture, the method comprising:
inserting one or more proximal pins into a patient's radius;
inserting one or more distal pins into a patient's carpal or metacarpal bone such that the fracture is positioned in-between the one or more proximal pins and the one or more distal pins;
coupling the one or more proximal pins and the one or more distal pins to a spanning rod, the spanning rod extending across the fracture;
inserting a K-wire into a fracture site including the fracture;
coupling an external fixation K-wire clamp to the spanning rod and to the inserted K-wire;
securing the K-wire to the external fixation K-wire clamp; and
removing, post-healing, the one or more distal pins, the one or more proximal pins, the K-wire, and the spanning rod.

18. The method of claim 17, wherein the one or more proximal pins are coupled to the spanning rod using a proximal rod-to-pin clamp and the one or more distal pins are coupled to the spanning rod using a distal rod-to-pin clamp.

19. The method of claim 17, further comprising securing a second K-wire to the external fixation K-wire clamp such that it is orientated non-parallel to the first K-wire.

20. The method of claim 19, wherein the first and second K-wires are not both in a plane parallel to the spanning rod.
